(19) United States
(12) Patent Application Publication
(10) Pub. No.: US 2020/0315547 A1
(43) Pub. Date: Oct. 8, 2020

(54) VERTEBRAL ARTERY DISSECTION RISK EVALUATION METHOD, COMPUTER DEVICE, AND STORAGE MEDIUM

(71) Applicant: TENCENT AMERICA LLC, Palo Alto, CA (US)

(72) Inventors: Zhen QIAN, Sunnyvale, CA (US); Hui TANG, Mountain View, CA (US); Nan DU, Santa Clara, CA (US); Min TU, Cupertino, CA (US); Kun WANG, San Jose, CA (US); Lianyi HAN, Palo Alto, CA (US); Wei FAN, New York, NY (US)

(73) Assignee: TENCENT AMERICA LLC, Palo Alto, CA (US)

(21) Appl. No.: 16/372,780

(22) Filed: Apr. 2, 2019

(51) Int. Cl. A61B 5/00; A61B 5/055; A61B 5/021; G06T 7/11; G06T 7/149

(52) U.S. Cl. CPC A61B 5/7275; A61B 5/055; A61B 5/7267; A61B 5/02444; A61B 5/02125; G06T 7/11; G06T 7/149; A61B 5/004

(57) ABSTRACT

Method and apparatus for vertebral artery dissection risk analysis using hemodynamic variable based four dimensional magnetic resonance flow imaging, comprising obtaining four-dimensional phase-contrast magnetic resonance imaging data, performing pre-processing of the four-dimensional phase-contrast magnetic resonance imaging data, obtaining at least one blood hemodynamic marker from the four-dimensional phase-contrast magnetic resonance imaging data, classifying the at least one blood hemodynamic marker as a hemodynamic predictor of vertebral artery dissection, and creating a comprehensive risk evaluation of vertebral artery dissection using the hemodynamic predictor.

### BACKGROUND

### Field

The disclosed subject matter relates to diagnosis and risk evaluation of vertebral artery dissection (VAD).

### Description of Related Art

A VAD is a flap-like tear of the inner lining of the vertebral artery, which is located in the neck and supplies blood to the brain. VAD typically results in blood entering the arterial wall, which in turn may form a blood clot, thickening the artery wall and often impeding blood flow.

Although incidents of VAD are relatively few in the general population (affecting about 1.1 per 100,000 as reported in [1] Schievink W I, Roiter V. Epidemiology of cervical artery dissection, Front Neurol Neurosci. 2005; 20: 12-15, incidents have been rising in the past few decades. Further, VAD may be a primary cause of ischemic stroke in young and middle-aged populations. Moreover, a fairly large percentage of patients who suffer a VAD (approximately 67%-85% of all VAD cases) have a resulting permeant condition or suffer a stroke, as disclosed in [2] Kim Y-K, Schulman S. Cervical artery dissection: pathology, epidemiology and management, Thromb Res. 2009; 123: 810-821.

VAD can be classified into two types: spontaneous VAD and traumatic VAD. Spontaneous VAD generally originates from intrinsic factors, specifically a variety of underlying arteriopathies. Traumatic VAD on the other hand is generally caused by extrinsic factors, such as an injury to the neck. However the cause of a VAD incident can be combinative. That is, Patients with weak vertebral arteries can be more susceptible to extrinsic trauma and traumatic VAD.

Studies, such as [3] Debette S, Leys D. Cervical-artery dissections: predisposing factors, diagnosis, and outcome, Lancet Neurol, 2009; 8: 668-678, have shown possible associations between spontaneous VAD and a number of underlying diseases that can cause weaknesses in the vertebral arterial wall. These diseases include connective tissue diseases, inflammation, and certain types of genetic disorders. However, as noted in [4] Debette S, Markus H S, the genetics of cervical artery dissection: a systematic review, Stroke. 2009; 40: e459-66, none of these possible associations have been convincingly proven.

Vascular imaging techniques, such as computed tomography angiography (CTA) and magnetic resonance angiography (MRA), have typically been used for the diagnosis of VAD. However, so far to date, such imaging techniques have not been used or proposed to be used as a predictive tool for assessing vulnerabilities of the vertebral artery and for evaluating risks of developing future VAD in asymptomatic patients.

As noted above, recent studies have identified a number of potential contributors to the occurrence of VAD. Most of these risk factors are associated with underlying arteriopathies that may lead to vulnerabilities in the vertebral arteries. Such arteriopathies include, but are not limited to hereditary connective tissue diseases, fibromuscular dysplasia, and arterial inflammation. These risk factors have been respectively studied in [5] Rouviére S, Michelini R, Sarda P, Pagés M., Spontaneous carotid artery dissection in two siblings with osteogenesis imperfecta, Cerebrovasc Dis. 2004; 17: 270-272, Cervical-artery dissections: predisposing factors, diagnosis, and outcome (cited above), and [2] Cervical artery dissection: pathology, epidemiology and management (cited above).

Recently, ultrasound imaging has been used to assess arterial wall weakness in carotid arteries, which has been associated with the occurrence of carotid artery dissection, as explained in [6] Calvet D, Boutouyrie P, Touze E, Laloux B, Mas J-L, Laurent S. Increased stiffness of the carotid wall material in patients with spontaneous cervical artery dissection, Stroke. 2004; 35: 2078-2082.

Contrast-enhanced magnetic resonance imaging (MRI) has been used as a follow-up imaging tool post occurrence of a VAD incident, as disclosed in [7] Provenzale J M. MRI and MRA for evaluation of dissection of craniocerebral arteries, lessons from the medical literature. Emerg Radiol, 2009; 16: 185-193.

However, despite the use of these known techniques, there lacks an imaging-based predictive tool to evaluate the risks of VAD for prevention thereof.

The state of the art of four-dimensional phase-contrast magnetic resonance imaging (4D PC-MRI) will now be described.

Disturbed blood flow to the brain has been associated with several neurological diseases, from stroke and vascular diseases to Alzheimer's and cognitive decline.

Two dimensional phase-contrast magnetic resonance imaging (2D PC-MRI) has been clinically used to study blood flow in selected transverse planes of the cerebral arteries as disclosed in [8] Lotz J, Döker R, Noeske R, Schüttert M, Felix R, Galanski M, et al, In vitro validation of phase-contrast flow measurements at 3 T in comparison to 1.5 T: precision, accuracy, and signal-to-noise ratios. J Magn Reson Imaging. 2005; 21: 604-610.

In recent years, 4D PC-MRI has been developed and performed primarily in a research capacity to study time-resolved three dimensional (3D) blood flow in the aorta and carotid arteries, as discussed in [9] Stankovic Z, Allen B D, Garcia J, Jarvis K B, Markl M. 4D flow imaging with MRI, Cardiovasc Diagn Ther. 2014; 4: 173-192.

Due to their smaller size, vertebral arteries are not often targeted for 4D flow imaging. Indeed it is traditionally difficult to target these arteries for 4D flow imaging.

Moreover, because of the difficulties in the acquisition and post-processing of 4D PC-MRI, 4D PC-MRI is rarely used clinically.

Further, 4D PC-MRI has not yet been used to evaluate the risks of the occurrence of VAD.

### CITATION LIST

[1]: Schievink W I, Roiter V. Epidemiology of cervical artery dissection, Front Neurol Neurosci, 2005; 20: 12-15;
[2]: Kim Y-K, Schulman S. Cervical artery dissection: pathology, epidemiology and management, Thromb Res. 2009; 123: 810-821;
[3]: Debette S, Leys D. Cervical-artery dissections: predisposing factors, diagnosis, and outcome. Lancet Neurol, 2009; 8: 668-678;
[4]: Debette S, Markus H S, The genetics of cervical artery dissection: a systematic review, Stroke. 2009; 40: e459-66;
[5]: Rouviére S, Michelini R, Sarda P, Pagés M., Spontaneous carotid artery dissection in two siblings with osteogenesis imperfecta, Cerebrovasc Dis. 2004; 17: 270-272;
[6]: Calvet D, Boutouyrie P, Touze E, Laloux B, Mas J-L, Laurent S. Increased stiffness of the carotid wall material in patients with spontaneous cervical artery dissection, Stroke. 2004; 35, 2078-2082;
[7]: Provenzale J M. MRI and MRA for evaluation of dissection of craniocerebral arteries: lessons from the medical literature, Emerg Radiol. 2009; 16: 185-193;
[8]: Lotz J, Döker R, Noeske R, Schatert M, Felix R, Galanski M, et al., In vitro validation of phase-contrast flow measurements at 3 T in comparison to 1.5 T: precision, accuracy, and signal-to-noise ratios, J Magn Reson Imaging. 2005; 21: 604-610;
[9]: Stankovic Z, Allen B D, Garcia J, Jarvis K B, Markl M. 4D flow imaging with MRI, Cardiovasc Diagn Ther. 2014; 4: 173-192.

### Effects and Advantages of Certain Embodiments

The instant disclosure has been developed in light of the above circumstances.

Certain embodiments provide a predictive tool for evaluating the risk of developing VAD in certain high-risk asymptomatic patients. This and other embodiments may utilize 4D PC-MRI to visualize and quantify blood flow in the vertebral arteries. In certain embodiments, the time-resolved 3D flow velocity field extracted from the 4D PC-MRI may be processed to extract various hemodynamic variables, such as, for example, pulsatile wave velocity and arterial wall shear stress, to assess the healthiness of vertebral arteries and dynamic interactions between the vertebral arterial wall and blood flow.

In certain embodiments, the hemodynamic variables, may include, but are no way limited to the 4D flow velocity field, flow pulsatile velocity, and time-resolved distribution of the wall shear stress. These illusory hemodynamic variables may be concatenated to form a complex hemodynamic profile of a patient's vertebral arteries.

In certain embodiments, this profile may be used to train the aforementioned predictive tool using machine learning.

Also, in certain embodiments, the flow-imaging-based hemodynamic variables may be further integrated with the other imaging/laboratory/genetic test data, for example, to generate a comprehensive predictive tool for VAD. Further, in certain embodiments, such an integration may be performed through statistical analysis and/or machine learning.

That is, certain embodiments of the instant disclosure provide for a risk evaluation tool for VAD based on analysis of the hemodynamics in the vertebral arteries using 4D PC-MRI and the optional integration of the hemodynamic variables with other anatomic/clinical information.

Accordingly, certain embodiments of the instant disclosure provide for a method, apparatus, and storage medium for imaging time-resolved 3D flow fields with high spatial resolution in relatively small vessels, post-processing of 4D PC-MRI with minimal user interaction, non-invasive assessment of the healthiness and vulnerability of the vertebral arteries of an individual using flow imaging, and information fusion of multi-modality and multi-source data for the evaluation of VAD risks.

### SUMMARY

One or more embodiments provide a sememe prediction method, a computer device, and a storage medium.

According to an aspect of an embodiment, there is provided a method for vertebral artery dissection risk evaluation that includes obtaining four-dimensional phase-contrast magnetic resonance imaging data, performing pre-processing of the four-dimensional phase-contrast magnetic resonance imaging data, obtaining at least one blood hemodynamic marker from the four-dimensional phase-contrast magnetic resonance imaging data, a classifying the at least one blood hemodynamic marker as a hemodynamic predictor of vertebral artery dissection, and creating a comprehensive risk evaluation of vertebral artery dissection using the hemodynamic predictor.

According to an aspect of an embodiment, there is provided an apparatus for vertebral artery dissection risk evaluation that includes at least one memory configured to store computer program code; at least one hardware processor configured to access said computer program code and operate as instructed by said computer program code, said computer program code including: first obtaining code configured to cause said at least one hardware processor to obtain four-dimensional phase-contrast magnetic resonance imaging data, pre-processing code configured to cause said at least one hardware processor to perform pre-processing of the four-dimensional phase-contrast magnetic resonance imaging data, second obtainment code configured to cause said at least one hardware processor to obtain at least one blood hemodynamic marker from the four-dimensional phase-contrast magnetic resonance imaging data, classification code configured to cause said at least one hardware processor to classify the at least one blood hemodynamic marker as a hemodynamic predictor of vertebral artery dissection, and creation code configured to cause said at least one hardware processor to create a comprehensive risk evaluation of vertebral artery dissection using the hemodynamic predictor.

According to an aspect of an embodiment, there is provided a non-transitory computer-readable medium storing instructions for vertebral artery dissection risk evaluation, the instructions comprising: one or more instructions that, when executed by one or more processors of a device, cause the one or more processors to: obtain four-dimensional phase-contrast magnetic resonance imaging data, pre-process the four-dimensional phase-contrast magnetic resonance imaging data, obtain at least one blood hemodynamic marker from the four-dimensional phase-contrast magnetic resonance imaging data, classify the at least one blood hemodynamic marker as a hemodynamic predictor of vertebral artery dissection, and create a comprehensive risk evaluation of vertebral artery dissection using the hemodynamic predictor.

### BRIEF DESCRIPTION OF THE DRAWINGS

FIG. 1 is a diagram of an environment in which methods, apparatuses and systems described herein may be implemented, according to embodiments.

FIG. 2 is a diagram of example components of one or more devices of FIG. 1.

FIG. 3 is a diagram of a VAD diagnosis and risk evaluation method, according to embodiments.

FIG. 4 is a diagram of an aortic flow.

FIG. 5 is a flowchart illustrating a VAD diagnosis and risk evaluation, according to embodiments.

### DETAILED DESCRIPTION

To make the objectives, technical solutions, and advantages of this application be more clear and comprehensible, embodiments will be further described in detail with reference to the accompany drawings. It should be understood that, the specific implementations described herein are only used for interpreting this application, rather than limiting this application.

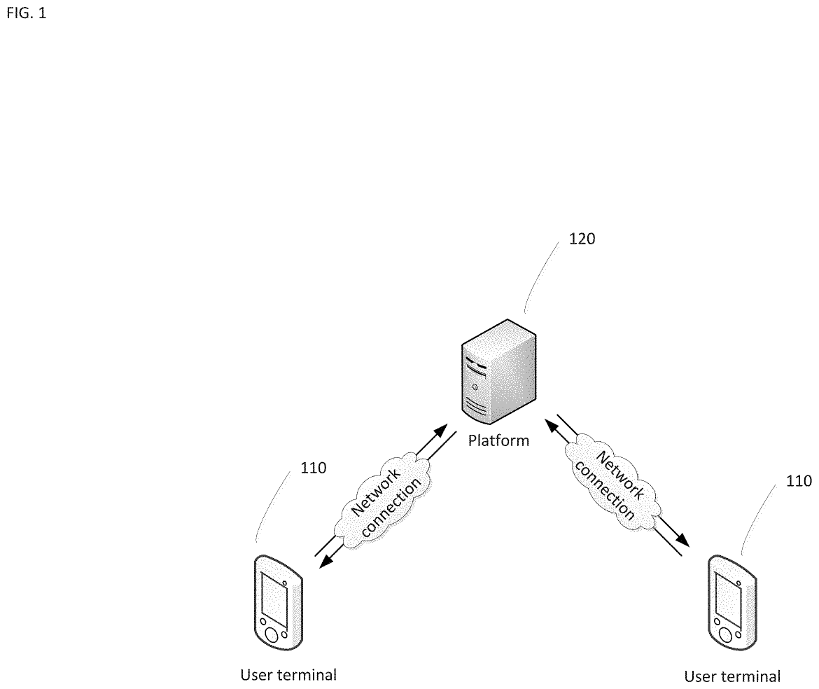

FIG. 1 is a schematic diagram of an application environment of a vertebral artery dissection (VAD) diagnosis and risk evaluation method according to an embodiment. As shown in FIG. 1, the application environment includes user terminals 110 and a server 120, and the user terminals 110 are in communication with the server 120. A user may enter data, for example four-dimensional phase-contrast magnetic resonance imaging (4D PC-MRI) data, patient data, and or hemodynamic variable data, through one of the user terminals 110, the entered data may be sent to the server 120 through a communications network, the server 120 may process the data, and provide information, based on the input data, relating to VAD diagnosis and risk prediction. Alternatively, the user may enter the data through one of the user terminals 110, the user terminal 110 may process the entered data, provide information, based on the input data, relating to VAD diagnosis and risk prediction, and send the information relating to VAD diagnosis and risk prediction to the server 120 through a communications network, which may in turn send the information relating to VAD diagnosis and risk prediction to the other of the user terminal 110.

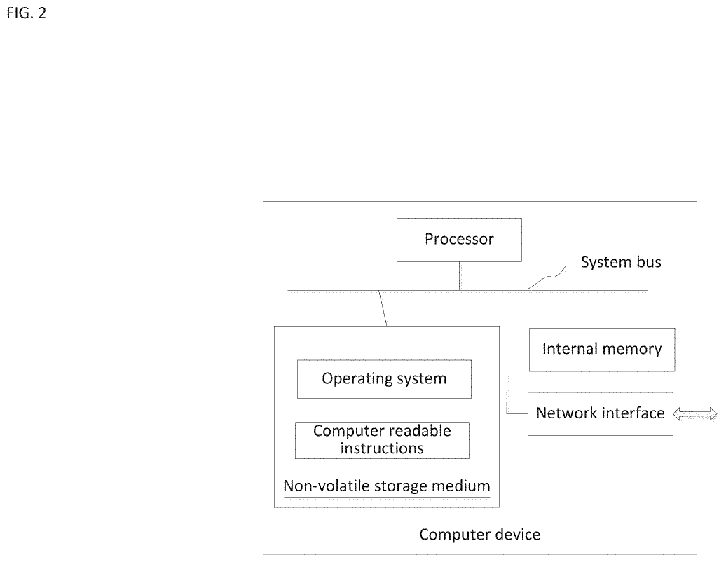

FIG. 2 is a schematic diagram of an internal structure of a computer device according to an embodiment. The computer device may be a user terminal or a server. As shown in FIG. 2, the computer device includes a processor, a memory, and a network interface that are connected through a system bus. The processor is configured to provide computation and control ability, to support operation of the computer device. The memory includes a non-volatile storage medium and an internal memory. The non-volatile storage medium may store an operating system and computer readable instructions, and the internal memory provides an environment for running the operating system and the computer readable instructions in the non-volatile storage medium. When the computer readable instructions are executed by the processor, the processor may perform a VAD diagnosis and risk prediction method. The network interface is configured to perform network communication with an external terminal.

Embodiments are not limited to the structure shown in FIG. 2, and various changes may be made without departing from the scope of the present disclosure. Specifically, the computer device may include more or less members than those in FIG. 2, or include a combination of two or more members, or include different member layouts.

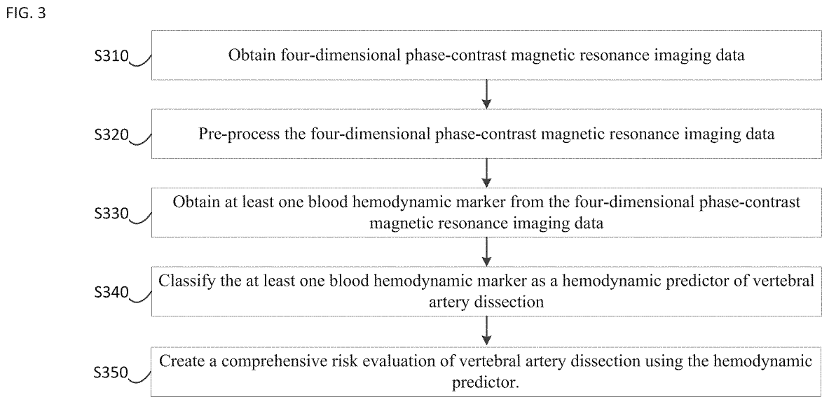

Referring to FIG. 3, in an embodiment, a VAD diagnosis and risk evaluation method is provided. The VAD diagnosis and risk evaluation method may be run in the server 120 shown in FIG. 1. The VAD diagnosis and risk evaluation method may include the following steps:

S310: Obtain four-dimensional phase contrast magnetic resonance image (4D PC-MRI) data.

Initially, it should be understood that prior to obtainment of the 4D PC-MRI data, a time-of-flight MR angiography (TOF MRA) or a contrast-enhanced MRA can be performed to serve as a localizer scan, in which a 3D region of interest (ROI) where vertebral arteries reside can be selected. Here, a larger ROI area may be selected to include other important cerebral arteries. However, a larger ROI may increase the scan time. The diameter of the vertebral arteries within the ROI can also be assessed using the localizer MRA, from which a minimal arterial diameter may be used to direct a setting of the spatiotemporal resolution of the PC-MRI. In certain embodiments, the transverse luminal area of the artery may cover enough voxels for a reliable quantification of flow velocity. In certain embodiments, the in-plane spatial resolution of the 4D PC-MRI may be set to 0.22×Diameter_min. The spatial resolution in the axial direction may be set to ≤2 mm. The temporal resolution may be set to <40 ms. The velocity encoding parameter (VENC) may be set to <150 cm/sec. In certain embodiments, 4D PC-MRI of the vertebral arteries may be performed with ECG-gating. The scan parameters of 4D PC-MRI may be determined based on considerations of both image quality and total scan time. When Gadolinium-based MRI contrast is used, for example, in certain embodiments, performing 4D flow imaging after the contrast-enhanced studies can improve the blood-to-tissue contrast and the velocity-to-noise ratio in the 4D PC-MRI images. When available, imaging acceleration methods may be used to shorten the acquisition time and improve the image quality.

S320: Pre-process the four-dimensional phase-contrast magnetic resonance imaging data. In this step, preprocessing is carried out on the obtained 4D PC-MRI data.

A number of sources may contribute to flow quantification errors in raw 4D PC-MRI data. While some sources of these errors may be compensated and corrected automatically on an MRI scanner, for example, typically, there are two phase errors that are addressed in the pre-processing S320 step.

First, the background phase offset induced by eddy currents is compensated. In certain embodiments, regions of static tissues in the 4D flow image, obtained from the 4D PC-MRI data, is identified using thresholding methods. Additionally, or in the alternative, a user can estimate the eddy currents-induced background phase offset errors using polynomial fitting, and subsequently remove the phase offset from the 4D flow data.

Second, phase correction is performed, if necessary, for example, when phase aliasing occurs. Certain embodiments may employ one or several phase-unwrapping algorithms.

In addition to correcting background phase offset and performing phase correction, the pre-processing step S320 may also include segmentation and tracking of the target arteries in certain embodiments. For instance, in some embodiments, flow path-line tracing may be performed only within the boundary of the artery lumen. In some embodiments, arteries in the magnitude image of the PC-MRI are segmented and tracked. Also, in some embodiments, automated segmentation of the arteries may be performed by first tracing the arterial centerlines and then performing the lumen segmentation using deformable models with a tubular shape.

In certain embodiments, not necessarily illustrated in FIG. 3, visualization techniques may be applied to visualize the 4D flow image in the vertebral arteries. In some embodiments, these visualization techniques may include, but are in no way limited to flow velocity vector maps, 3D streamlines, and time-resolved 3D path-lines.

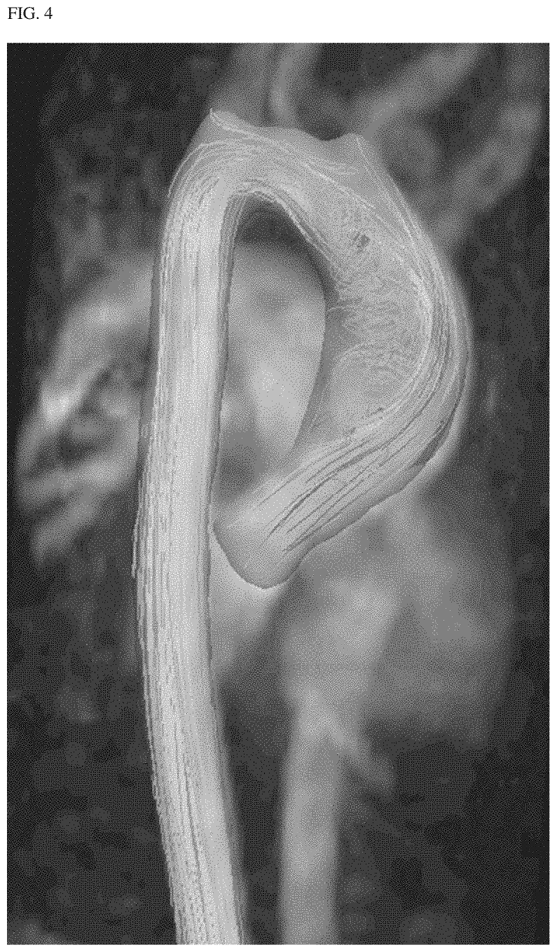

FIG. 4 illustrates a 3D streamlines visualization of a 4D aortic flow in a patient with bicuspid aortic valve (BAV). The darker color represents the flow velocity and the orientation of the line represents the flow direction. As shown in FIG. 4, in the thoracic segment of the aorta, flow visualization using the 3D streamlines techniques provides rich information of an arterial flow pattern, such as the locations and the velocities of the high speed jet and the helical flow. In addition, vessel narrowing, increased flow velocity, and increased pressure gradient can also be visualized using the 3D streamlines.

Referring back to FIG. 3, attention is brought to S330: Obtaining at least one blood hemodynamic marker from the four-dimensional phase-contrast magnetic resonance imaging data.

Since 4D PC-MRI provides full volumetric coverage of the ROI, the vertebral artery dissection (VAD) diagnosis and risk evaluation method provides an unique option of retrospective selection of 2D slices or 3D sub-regions in the 3D field of view for 3D flow quantification and analysis. Thus, besides conventional 2D flow parameters, such as, for example, transvalvular gradient and peak flow velocity, a number of advanced 4D blood hemodynamic markers can be harvested from the 4D PC-MRI image data. Some of these advanced markers are discussed below. However, it should be understood that the markers are not limited to those discussed below.

Shear rate (SR) may be calculated as a spatial gradient of the flow velocity field. It may be associated with the blood thrombus process because it is associated with forces experienced by blood components such as platelets and red blood cells.

Wall shear stress (WSS) is the friction force blood flow exerts on the vertebral arterial wall. It can be estimated in certain embodiments by taking the derivative of 4D flow velocity near the vessel wall boundary. WSS is believed to play an important role in the regulation of the functions of the endothelial cell and the extracellular matrix in the vessel wall. For example, low WSS has been associated with the development of atherosclerosis, and high WSS has been associated with vessel dilation and the formation of aneurysms.

Pulse wave velocity (PWV) is the propagation speed of the systolic wave front through the artery. It is a direct indicator of arterial wall stiffness and an important predictor of arteriopathy progression in patients with hypertension and connective tissue diseases. In order to automatically measure PWV in a 4D flow image, in certain embodiments, velocity waveforms can be measured at selected sites along the centerline of the vertebral artery. Then, PWV may be calculated as the ratio of the distance between measurement sites and the transit-time.

Flow eccentricity (FE) may lead to jet impingement on the vertebral artery wall, and may be associated with weakness in the vessel wall and the occurrence of VAD.

As noted above, it should be understood that the above advanced hemodynamic markers are not all of the advanced hemodynamic markers that may be obtained from the 4D PC-MRI image data. Other advanced hemodynamic markers derived from the 4D PC-MRI image data may include, but are in no way limited to turbulence, kinetic energy, energy dissipation, relative pressure fields, and flow displacement.

Accordingly in S330, at least one of these blood hemodynamic markers is obtained from the 4D PC-MRI image data.

It will be understood that the aforementioned methods of obtaining the aforementioned advanced hemodynamic markers are in no way limiting. Indeed, certain embodiments of the disclosure may obtain the aforementioned advanced hemodynamic markers in different manners.

S340: Classify the at least one blood hemodynamic marker as a hemodynamic predictor of vertebral artery dissection.

Here, the obtained advanced blood hemodynamic marker(s) are classified. In certain embodiments, this classification may be performed by deep learning. However, other classification methods may also be used.

Additionally, when other parameters (e.g. not necessarily advanced blood hemodynamic markers) are available, such as, for example, artery geometric measurements derived from the contrast-enhanced CTA or MRA, patient clinical and medical information, laboratory test results, genetic test results, and potential risk level of the extrinsic factors related to VAD, these additional parameters may also be classified. In certain embodiments, these additional parameters may be classified using deep learning. However, other classification methods may also be used.

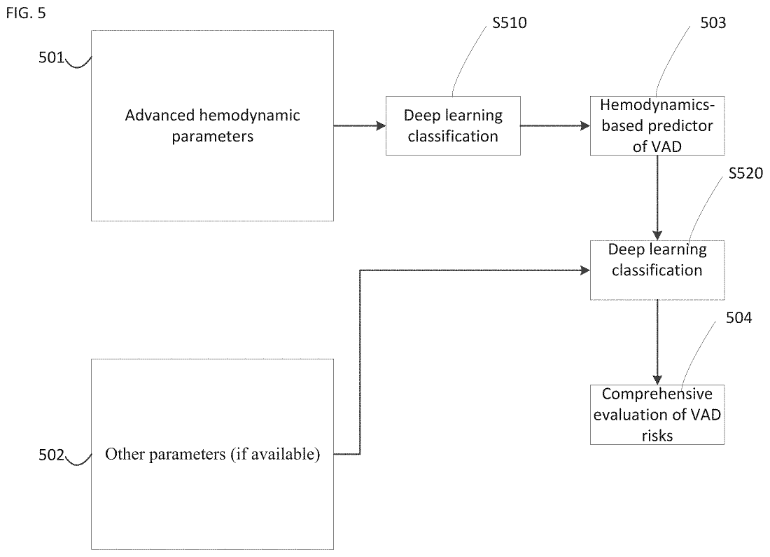

This classification process is illustrated in more detail in FIG. 5, which presents an exemplary view of a certain embodiment. As shown in FIG. 5, 501 corresponds to the advanced hemodynamic parameter(s) and 502 corresponds to the other parameter(s). When other parameters are not available, for example, in certain embodiments, the advanced hemodynamic parameter(s) are classified using deep learning in S510, resulting in hemodynamics-based predictor(s) 503. Hemodynamics-based predictor(s) 503 may then be further classified in certain embodiments in S520, so as to produce a comprehensive evaluation of VAD risks 504. This comprehensive evaluation of VAD risks will be described later in more detail with reference again to FIG. 3.

In the embodiment depicted in FIG. 5, When other parameters 502 are available, they may be classified, in certain embodiments in S520, and used together with the classified advanced hemodynamic parameter(s) 501 and/or the classified hemodynamics-based predictor(s) 503 to produce the comprehensive evaluation of VAD risks 504.

Referring again to FIG. 3, in S350, a comprehensive evaluation of VAD risks 504 is creating using the hemodynamic predictor.

The above described method uses 4D PC-MRI flow imaging to extract hemodynamic information that is closely related to the healthiness of the vertebral arteries. The discussed embodiments achieves the following functions:

Acquisition of high-resolution 4D PC-MRI image data in the vertebral arteries. Embodiments may provide a guideline to achieve high-resolution 4D PC-MRI image data of the cerebral and extracerebral vessels using commercial MRI scanners.

Post-processing of 4D PC-MRI image data with minimal user interactions for the extraction of the time-resolved 3D flow velocity in the vertebral arteries, in certain embodiments.

Extraction of in-vivo hemodynamic variables that are associated with vulnerability of the vertebral arterial wall, in certain embodiments. A variety of hemodynamic parameters related to arteriopathy may be identified and extracted from the 4D PC-MRI image data in certain embodiments.

Study the healthiness of the vertebral arterial wall in vivo using machine learning techniques with the hemodynamic variables as the input features, as discussed above with reference to certain embodiments.

Integration of hemodynamic information and other imaging/clinical/laboratory/genetic testing results for the comprehensive risk evaluation of VAD, as discussed above with reference to certain embodiments.

In certain high-risk asymptomatic populations, which are prone to either intrinsic, extrinsic, or both factors of VAD risk, identifying patients with an underlying vertebral arteriopathy and advising proactive prevention of VAD may be beneficial. For instance, patients with family histories of spontaneous arterial dissection could benefit from a risk-evaluation test for VAD. Also, for athletes in competitive sports, such a screening tool would be much needed by both the athlete community and the sports industry.

Certain embodiments of the instant disclosure provide for the evaluation of other relatively smaller and deeper arteries (e.g., diameter range: 3-5 mm; not easily accessible by ultrasound).

Also, the above-described embodiments may alternative, or combinatively be modified as follows.

The afore-described classifications may be replaced by other machine learning-based or statistics-based methods, that are not necessarily rooted in deep learning. This may be especially true in embodiments, for which very limited training data is available.

Segmentation of the vertebral arteries in the magnitude image of the 4D PC-MRI may be performed by using other segmentation methods, such as the 3D levelset method.

In embodiments where training data is limited, for classifying the parameters, mean values may be used. Another approach would be to treat the missing data as hidden variables, and use an EM algorithm to estimate them.

The VAD diagnosis and risk evaluation apparatus/method corresponds to the other of the VAD diagnosis and risk evaluation apparatus/method, and the specific technical features that correspond are not repeated herein.

A person of ordinary skill in the art may understand that all or some of the modules, units, components and procedures of the foregoing embodiments may be implemented by a computer program instructing relevant hardware. The program may be stored in a non-volatile computer readable storage medium. When the program is executed, the program may control the hardware to execute the procedures of the embodiments of each foregoing method. Any usage of a memory, storage, a database or other media in each embodiment of this application may include non-volatile and/or volatile memories. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. For description, rather than for limitation, RAM may be in various forms, for example, a static RAM (SRAM), a dynamic RAM (DRAM), a synchronous DRAM (SDRAM), a double data rate SDRAM (DDRSDRAM), an enhanced SDRAM (ESDRAM), a Synchlink DRAM (SLDRAM), a Rambus direct RAM (RDRAM), a directly memory bus dynamic RAM (DRDRAM), and a memory bus dynamic RAM (RDRAM).

Each technical feature in the foregoing embodiments may be combined randomly. For simplified description, not all possible combinations of each technical feature in the foregoing embodiments are described. However, the combinations of the technical features shall be considered to fall within the scope of the specification as long as the combinations are not contradictory. The foregoing embodiments only describe several implementations of this application, and their description is specific and detailed, but cannot therefore be construed as a limitation to the patent scope of the present disclosure. It should be noted that a person of ordinary skill in the art may further make variations and improvements without departing from the conception of this application, and these all fall within the protection scope of this application. Therefore, the patent protection scope of this application should be subject to the appended claims.

What is claimed is:

1. A method, performed by at least one computer processor, the method comprising:
obtaining four-dimensional phase-contrast magnetic resonance imaging data,
performing pre-processing of the four-dimensional phase-contrast magnetic resonance imaging data,
obtaining at least one blood hemodynamic marker from the four-dimensional phase-contrast magnetic resonance imaging data,
classifying the at least one blood hemodynamic marker as a hemodynamic predictor of vertebral artery dissection, and
creating a comprehensive risk evaluation of vertebral artery dissection using the hemodynamic predictor.

2. The method of claim 1, wherein the classifying of the at least one blood hemodynamic marker as a hemodynamic predictor of vertebral artery dissection is performed using deep learning.

3. The method of claim 1, wherein the comprehensive risk evaluation of vertebral artery dissection is created by using at least one of the following additional parameters: artery geometry, patient age, patient sex, patient race, medical records, laboratory test results, genetic test results, and extrinsic trauma factors.

4. The method of claim 3, wherein the at least one additional parameter is classified as a predictor of vertebral artery dissection using deep learning.

5. The method of claim 1, the method further comprising performing localized scanning prior to obtaining the four-dimensional phase-contrast magnetic resonance imaging data, the performance of the localized scanning comprising selecting a three-dimensional region of interest of vertebral arteries.

6. The method of claim 1, wherein the at least one blood hemodynamic marker is a four dimensional flow velocity, a shear rate, a wall shear stress, a pulse wave velocity, or a flow eccentricity.

7. The method of claim 3, wherein the at least one blood hemodynamic marker is a four dimensional flow velocity, a shear rate, a wall shear stress, a pulse wave velocity, or a flow eccentricity.

8. The method of claim 1, wherein the classifying of the at least one blood hemodynamic marker as a hemodynamic predictor of vertebral artery dissection is performed using machine learning or statistics based learning.

9. The method of claim 1, the method further comprising performing segmentation and tracking prior to obtaining the four-dimensional phase-contrast magnetic resonance imaging data.

10. The method of claim 9, wherein the segmentation and tracking is performed by first tracing arterial centerlines and then performing lumen segmentation using deformable models with a tubular shape.

11. An apparatus, comprising:
at least one memory configured to store computer program code;
at least one hardware processor configured to access said computer program code and operate as instructed by said computer program code, said computer program code including:
first obtaining code configured to cause said at least one hardware processor to obtain four-dimensional phase-contrast magnetic resonance imaging data,
pre-processing code configured to cause said at least one hardware processor to perform pre-processing of the four-dimensional phase-contrast magnetic resonance imaging data,
second obtainment code configured to cause said at least one hardware processor to obtain at least one blood hemodynamic marker from the four-dimensional phase-contrast magnetic resonance imaging data,
classification code configured to cause said at least one hardware processor to classify the at least one blood hemodynamic marker as a hemodynamic predictor of vertebral artery dissection, and
creation code configured to cause said at least one hardware processor to create a comprehensive risk evaluation of vertebral artery dissection using the hemodynamic predictor.

12. The device of claim 11, wherein the classification code is configured to cause said at least one hardware processor to classify the at least one blood hemodynamic marker as a hemodynamic predictor of vertebral artery dissection, using deep learning.

13. The device of claim 11, wherein the creation code is configured to cause said at least one hardware processor to create the comprehensive risk evaluation of vertebral artery dissection using at least one of the following additional parameters: artery geometry, patient age, patient sex, patient race, medical records, laboratory test results, genetic test results, and extrinsic trauma factors.

14. The device of claim 13, wherein the classification code is further configured to classify the at least one additional parameters as a predictor of vertebral artery dissection using deep learning.

15. The device of claim 11, wherein the at least one blood hemodynamic marker is a four dimensional flow velocity, a shear rate, a wall shear stress, a pulse wave velocity, or a flow eccentricity.

16. The device of claim 13, wherein the at least one blood hemodynamic marker is a four dimensional flow velocity, a shear rate, a wall shear stress, a pulse wave velocity, or a flow eccentricity.

17. The device of claim 11, wherein the classification code is configured to cause said at least one hardware processor to classify the at least one blood hemodynamic marker as a hemodynamic predictor of vertebral artery dissection, using machine learning or statistics based learning.

18. The device of claim 11, the device further comprising segmentation and tracking code configured to cause said at least one hardware processor to segment and track the four-dimensional phase-contrast magnetic resonance imaging data.

19. The device of claim 18, wherein the segmentation and tracking code is configured to cause said at least one hardware processor to segment and track the four-dimensional phase-contrast magnetic resonance imaging data by first tracing arterial centerlines and then performing lumen segmentation using deformable models with a tubular shape.

20. A non-transitory computer-readable medium storing instructions, the instructions comprising: one or more instructions that, when executed by one or more processors of a device, cause the one or more processors to:
obtain four-dimensional phase-contrast magnetic resonance imaging data,
pre-process the four-dimensional phase-contrast magnetic resonance imaging data,
obtain at least one blood hemodynamic marker from the four-dimensional phase-contrast magnetic resonance imaging data,
classify the at least one blood hemodynamic marker as a hemodynamic predictor of vertebral artery dissection, and
create a comprehensive risk evaluation of vertebral artery dissection using the hemodynamic predictor.
